(19) United States
(12) Patent Application Publication
(10) Pub. No.: US 2005/0239844 A1
(43) Pub. Date: Oct. 27, 2005

(54) Composition fo preventing secretion of immunoglobulin e-dependent histamine releasing factor

(75) Inventors: Kyung-Lim Lee, Seoul (KR); Chul-Hee Lee, Seoul (KR); Seung-Hee Choi, Seoul (KR)

Correspondence Address: GREENBLUM & BERNSTEIN, P.L.C., 1950 ROLAND CLARKE PLACE, RESTON, VA, 20191, US

(73) Assignee: KYUNG-LIM LEE, DAEHYEON-DONG SEODAEMOON-KU, 120-750 (RU)

(21) Appl. No.: 10/533,077

(22) PCT Filed: Nov. 3, 2003
(86) PCT No.: PCT/KR03/02332

§ 371 Date: Apr. 28, 2005

(30) Foreign Application Priority Data
Nov. 2, 2002 (KR) 10-2002-0067653
Oct. 28, 2003 (KR) 10-2003-0075511

(51) Int. Cl. A61K 31/4439

(52) U.S. Cl. 514/338

(57) ABSTRACT

The present invention relates to a composition for inhibiting the secretion of an immunoglobulin E (IgE)-dependent histamine-releasing factor, and pharmaceutical use thereof. The composition of the present invention contains a benzimidazolic compound having proton pump inhibitor activity, as an active ingredient. Furthermore, the composition of the present invention may contain at least one substance selected from the group consisting of fenoctimine, oleic acid, catechin, scopadulciol, pentagalloyl glucose, bufalin, and macrolide antibiotic bafilomycin and concanamycin, all having proton pump inhibitor activity, as an additional active ingredient or a single active ingredient. The composition of the present invention can be advantageously used as a pharmaceutical composition for the prevention and treatment of various allergic diseases and malaria, which are caused by the IgE-dependent histamine-releasing factor.

### TECHNICAL FIELD

The present invention relates to a composition for inhibiting the secretion of an immunoglobulin E (IgE)-dependent histamine-releasing factor (hereinafter, referred to as “HRF”), and pharmaceutical use thereof.

### BACKGROUND ART

HRF is known to perform an important role in late phase allergic reaction (hereinafter, referred to as “LPR”), and to stimulate a basophil to induce histamine secretion so as to mediate various allergic diseases (McDonald et al., 1995).

Also, HRF is known to be a kind of tumor protein that binds to Artemisinin as an anti-malaria agent in malaria mosquitoes (Bhisutthibhan, 1998).

Allergic diseases, which are caused by HRF, include anaphylaxis, asthma, urticaria, allergic rhinitis, allergic bronchiectasis, hay fever, atopic dermatitis and the like. Particularly, as the probability of exposure to various allergens rapidly increases due to environmental contamination, the allergic diseases show a tendency to further increase.

HRF is hydrophilic in nature and a cytoplasmic protein having no signal peptide that is generally contained in proteins which are released extracellularly. However, a study result showing that HRF is also detected extracellularly was reported (McDonald et al., 1995), and HRF was found to stimulate an IgE-sensitized basophil extracellularly to induce histamine secretion.

Accordingly, there have been efforts in an attempt to establish a mechanism on the extracellular secretion of the cytoplasmic protein HRF and to develop a substance capable of inhibiting this process, and thus, to prevent and inhibit various diseases caused by HRF.

Molecules that were known, up to now, to have no signal peptide while being detected extracellularly, like HRF, include a basic fibroblast growth factor (FGF-2), a plasminogen activator inhibitor 2 (PAI-2), a carbohydrate-binding protein 30 (CBP 30) and the like.

Although the secretion pathway of such molecules was not yet clearly established, a study result indicating that a Na/K-ATPase pump can be a potential FGF-2 release pathway was recently reported (Florkiewicz RZ, 1998).

On the basis of this fact, the present inventors have established the secretion pathway of HRF on the assumption that HRF can also be secreted via the pump, and invented a composition capable of inhibiting HRF secretion.

### DISCLOSURE OF INVENTION

Accordingly, an object of the present invention is to provide a composition for inhibiting HRF secretion and, pharmaceutical use thereof.

The present invention provides a composition for inhibiting the secretion of an IgE-dependent histamine-releasing factor, and pharmaceutical use thereof.

The composition of the present invention contains a benzimidazolic compound with proton pump inhibiting activity, as an active ingredient.

As used herein, the term “proton pump” means H⁺-K⁺ APase, a membrane protein, which actively transports a hydrogen ion (H⁺) to the outside of cytoplasm, in a direction opposite to concentration gradient.

The benzimidazolic compound, which is used in the inventive composition, is a lipid-soluble weak base consisting of a benzimidazole ring, a pyridine ring and a methylsulfinyl group located between them, and has a structure represented by the following formula (1): 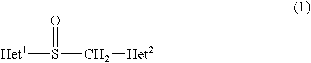
wherein Het¹ is 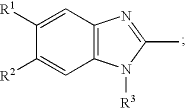
   Het² is 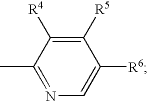
   R¹ and R² which may be the same or different, each independently represents hydrogen, methoxy, or difluoromethoxy; R³ represents hydrogen or sodium; and R⁴, 5⁵ and R⁶, which may be the same or different, each independently represents hydrogen, methyl, methoxy, methoxypropoxy, or trifluoroethoxy.

The benzimidazolic compound used in the present invention may be preferably one or more selected from the group consisting of omeprazole, lansoprazole, pantoprazole and rabeprazole.

In addition to such substances, the benzimidazolic compound used in the present invention may also be derivatives and isomers of such substances, and preferably hydroxy-omeprazole, hydroxy-lansoprazole, carboxylic acid derivatives of omeprazole, desmethyl-pantoprazole, esomeprazole, or the like.

The benzimidazolic compound used in the present invention inhibits a proton pump so as to inhibit the extracellular secretion of HRF. This is because the proton pump is involved in the secretion process of HRF.

Thus, the use of the inventive composition can inhibit HRF secretion induced by allergy-causing substances resulting in an increase in histamine concentration.

Particularly, the composition of the present invention inhibits HRF secretion caused by C48/80 so as to reduce HRF or histamine concentration in blood, resulting in a remarkable reduction in the probability of death from the systemic anaphylaxis reaction caused by C48/80.

The composition of the present invention reduces the amount of HRF and histamine in blood, such that the probability of death from the systemic anaphylaxis reaction caused by allergy-causing substances is remarkably reduced and symptoms of allergic rhinitis are effectively mitigated.

Furthermore, the composition of the present invention has the effect of reducing the expression of symptoms and histological change of allergic rhinitis in a mouse model with allergic rhinitis.

Thus, the composition of the present invention can be advantageously used as a pharmaceutical composition for the prevention and treatment of various allergic diseases caused by HRF, i.e., asthma diseases, including asthma from foods or drugs, such as aspirin, allergic asthma, urticaria, anaphylaxis, allergic rhinitis, allergic bronchiectasis, hay fever, atopic dermatitis and malaria.

The composition of the present invention may contain a substance with the same or similar function to the above-mentioned substances, as an additional active ingredient or a single active ingredient. Specifically, the composition of the present invention may contain, as an additional active ingredient or a single active ingredient, at least one substance selected from the group consisting of fenoctimine, oleic acid, catechin, scopadulciol, pentagalloyl glucose, bufalin, and macrolide antibiotic bafilomycin and concanarnycin, which are known to have proton pump inhibition activity.

If necessary, the composition of the present invention may additionally contain a substance with a different function from the above-mentioned substances, as an active ingredient.

In addition to such active ingredients, the composition of the present invention may contain pharmaceutically suitable and physiologically acceptable adjuvants, such as solubilizers, solvents, disintegrants, sweeteners, binders, coating agents, swelling agents, lubricants, or flavoring agents.

For administration, the composition of the present invention may be formulated with at least one pharmaceutically acceptable carrier in addition to the above-mentioned active ingredients.

Examples of the pharmaceutically acceptable carrier, which can be used in the present invention, include saline solution, dextrose solution, maltodextrin solution, glycerol, ethanol and a mixture thereof. If necessary, the inventive composition may contain other conventional additives, such as antioxidants, buffer solution and bacteriostatic agents. Moreover, the inventive composition may be formulated into an injectable liquid, such as water solution, suspension and emulsion, pills, capsules, granules or tablets, by the addition of diluents, dispersants, surfactants, binders and lubricants. Furthermore, it can be preferably formulated depending on diseases or its components, using a method described in Remington's Pharmaceutical Science (latest edition), Mack Publishing Company, Easton Pa.

The formulated form of the inventive composition may be granules, powders, coated pills, tablets, capsules, suppositories, syrup, juice, suspension, emulsion, drops, injectable liquids, or a sustained release preparation of the active compounds.

The composition of the present invention can be administered by various routes, including oral, intravenous, intraarterial, intraabdominal, intramuscular, intrastemal, subcutaneous, intradermal, intranasal, topical, rectal, and intradermal routes, in a conventional manner.

When the composition of the present invention contains pantoprazole, it is preferably administered at a dosage of 10 mg/kg body weight for a rat or mouse, and at a dosage of 100 μg-1 mg per kg body weight for a man. The dosage of the inventive composition can vary depending on various factors, including the kind of diseases, the severity of diseases, the kind and content of the active ingredients and other components in the composition, the kind of a formulation, the age, body weight, general health condition, sex and diet of a patient, the administration time, administration route and percent secretion of the composition, therapeutic period, and a drug that is used simultaneously with the composition.

The composition of the present invention is a drug that has been previously used for the treatment of gastric ulcer, duodenal ulcer, Zollinger-Ellison syndrome and reflux esophagitis and whose safety was established. Thus, it has no particular side effects on a living body.

The composition of the present invention can be used alone or in combination with diet therapy, chemical therapy, and methods utilizing an agent for regulating biological reaction.

### BRIEF DESCRIPTION OF DRAWINGS

FIG. 1 shows that HRF is secreted into serum of a variety of allergic patients;

FIG. 2 shows that HRF is secreted extracellularly by allergy-causing substances;

FIG. 3 shows that a composition of the present invention inhibits HRF secretion, which is increased by allergy-causing substances in vitro;

FIG. 4 shown that a composition of the present invention reduces blood HRF concentration, which is increased by allergy-causing substances in vivo;

FIG. 5 shows that a composition of the present invention inhibits the expression of symptoms of allergic rhinitis in an allergic mouse model; and

FIG. 6 shows that a composition of the present invention inhibits the increase of eosinophil number in a mouse with allergic rhinitis.

### BEST MODE FOR CARRYING OUT THE INVENTION

The present invention will hereinafter be described in further detail by examples. It will, however, be obvious to a person skilled in the art that the examples are given for illustrative purpose only, and the present invention is not limited to or by the examples.

### EXAMPLE 1

### Effect of Inventive Composition on Inhibition of HRF Secretion

To examine the effect of the inventive composition on the inhibition of HRF secretion, the correlation between HRF secretion, allergic symptoms and allergy-causing substances was established and then the effect of the inventive composition on HRF secretion was examined.

1) Examination of correlation between HRF secretion, allergic symptoms and allergy-causing substances

The correlation between HRF secretion, allergic symptoms and allergy-causing substances was examined as follows.

1-1) In order to examine if the secretion of HRF in various allergic symptoms is increased, a variety of allergic patients, including patients showing asthma response to aspirin administration, patients showing asthma response to other allergens than aspirin, patients with allergic asthma, patients showing food-dependent exercise-induced anaphylaxis, and patients with urticaria, were selected and examined for an increase in HRF secretion.

The patients showing asthma response to aspirin administration were divided into two groups consisting of a dual response patient group showing asthma response both just and some time after aspirin administration, and an early response patient group showing asthma response only just after aspirin administration.

To examine if HRF is present in blood, a small amount of serum was collected from each patient, and in the case of the patients showing allergic response to other allergens than aspirin, serum was collected in a state where they were not exposed to the allergens. As a negative control group, the serum of normal persons who show no allergic response was also collected.

The serum of each group was mixed with the same amount of 4× sample buffer solution. At this time, the ratio of glycerol in the sample buffer solution was 50% such that the attraction phenomenon of bands upon electrophoresis is prevented. The sample mixture was denatured in boiling water for 3 minutes, cooled for a short time, centrifuged at 12,000 rpm for 5 minutes, and then only the supernatant was collected.

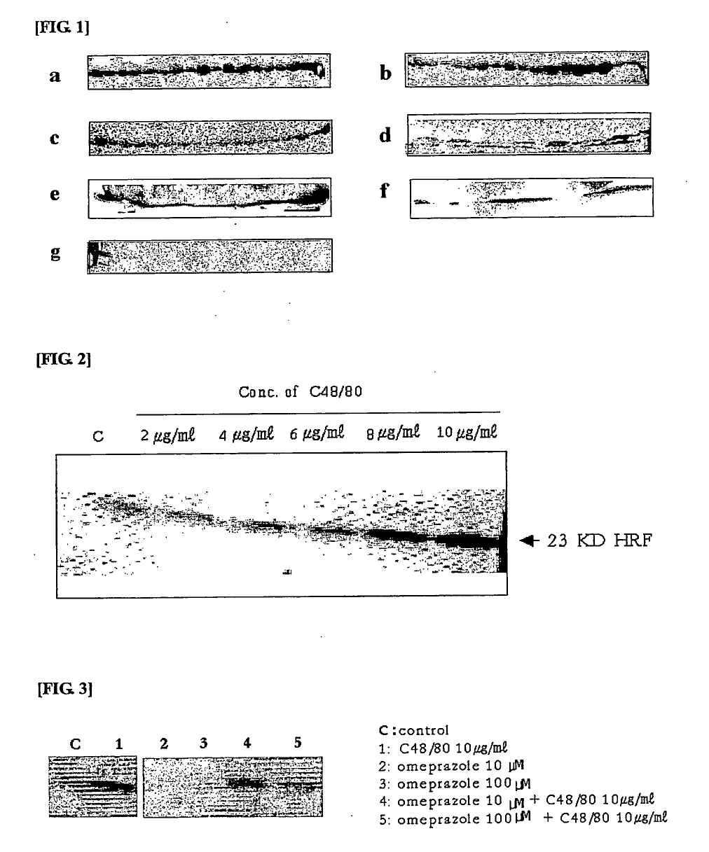

The sample prepared as described above was subjected to Western blotting analysis in the same manner as the part 1-2) of Example 1, and the results are shown FIG. 1.

As shown in FIG. 1, a high concentration of HRF was detected in all the serum of the patients with dual response to aspirin administration (a), the patients with early response to aspirin administration (b), the patients showing asthma response to other allergens than aspirin (c), the patients with allergic asthma (d), the patients with food-dependent exercise-induced anaphylaxis (e), and the patients with urticaria (f). On the other hand, in the serum of the normal person (g), HRF was no substantially detected.

Thus, since HRF is detected in a variety of allergic patients, it can be found that HRF secretion performs an important role in allergic reaction.

1-2) Increase of HRF secretion by C48/80

In order to examine the effect of allergy-causing substances on HRF secretion, C48/80 (compound 48/80, Sigma) as a typical allergy-causing substance was selected and subjected to the following test.

As a cell model for BRF secretion, a U-937 cell was selected and cultured in a RMPI 1640 medium containing 1% L-glutamine, 100 U/ml penicillin, 100 U/ml streptomycin and 10% fetal bovine serum, at 37° C. and 5% CO₂.

The culture medium of the U-937 cell was centrifuged at 1,000 rpm for 3 minutes, and the cell was collected, washed one time with a phosphate buffered saline (PBS; 200 mg/l potassium chloride, 200 mg/l potassium dihydrogen phosphate, 8 g/l sodium chloride, 1.15 g/l sodium phosphate dibasic), and centrifuged again at 1,000 rpm for 3 minutes, and the cells were collected.

The collected cells were added with a RPMI medium containing only 1% L-glutamine, and shaken such that the precipitated cells were well mixed with the medium. Then, some of the solution was taken and dyed with tryphan blue (Gibco BRL), and the number of living cells was counted. The cells were plated at 10⁶ cells/well in a 6-well plate, and left to stand in a starvation state at 37° C. and 5% CO₂ for 5 hours. Upon the end of the cell starvation, each well of the 6-well plate was treated with C48/80 (Sigma) at concentrations of 0 μg/ml, 2 μg/ml, 4 μg/ml, 6 μg/ml, 8 μg/ml and 10 μg/ml for 30 minutes, and the cells were separated from the supernatant.

The supernatant was added with a protease inhibitor cocktail consisting of 100 mM PMSF, 1 mg/ml aprotinin, 1 M benzamidine and 10 mg/ml leupeptin, and centrifuged at 4° C. and 12,000 rpm for several hours with a concentrator (Centricon, Sartorius) using the centrifugal force so as to concentrate to 1/100 of the original volume.

The concentrate was added with 4× sample buffer (1 M Tris (pH 6.8), 10% SDS, 100% glycerol, several drops of bromophenol blue, a small amount of β-mercaptoethanol, and deionized water), denatured in boiling water for 10 minutes, and then immediately, moved onto ice so as to prevent the denatured protein from being returned to the original state.

The sample obtained as described above was electrophoresed on 15% SDS-PAGE gel, transferred to a nitrocellulose membrane and then analyzed by Western blotting.

In the Western blotting analysis, a 4,000-fold dilution of a polyclonal anti-HRF antibody in Tris buffered saline with 0.05% tween 20 was used as a primary antibody, and a 10,000-fold dilution of a goat anti-rabbit IgG-HRP in Tris buffered saline with 0.05% Tween 20 was used as a secondary antibody. Then, the protein bands of the washed membrane were analyzed using an image analyzer system, and the results are shown in FIG. 2.

As shown in FIG. 2, the histamine-releasing substance C48/80 caused the increase of HRF secretion in a concentration-dependent manner. Due to this characteristic, C48/80 was used as an agent for increasing BRF secretion in a subsequent Example.

2) Preparation of Inventive Composition

The composition of the present invention was prepared by dissolving omeprazole (Sigma) in ethanol at a concentration of 13 mM.

When treating cells with the composition of the present invention, the omeprazole was used at final concentrations of 10 μM and 100 μM.

3) Effect of Inventive Composition on Inhibition of BF Secretion

The effect of the composition of the present invention on the inhibition of HRF secretion was examined as follows.

In the same manner as the part 1-2) of Example 1, cells were plated on a 6-well plate, and left to stand in a starvation state for 5 hours. Then, an untreated group, a group treated with 10 μg/ml of C48/80, a group treated with each of 10 μM and 100 μM of omeprazole, a group treated with 10 μM of omeprazole and 10 μg/ml of C48/80, and a group treated with 100 μM of omeprazole and 10 μg/ml of C48/80, were provided, and the cells of each group was isolated from the supernatant and collected.

The collected cells were washed one time with a phosphate buffered saline, precipitated by centrifugation at 12,000 rpm for 10 minutes, resuspended in a cell lysis buffer (20 mM Tris (pH 7.4), 150 mM sodium chloride, 5 mM EDTA, 0.5% deoxycholate, 2 mM EGTA, 1% NP40, protease cocktail), and vortexed.

The cell mixture was kept on ice with 2 or 3 times vortexing, and then centrifuged at 12,000 rpm for 10 minutes, and the supernatant was collected. A portion of the supernatant was taken and quantified for protein by a Bradford assay. Then, the supernatant containing the same amount of protein was added with a 4× sample buffer, boiled for 10 minutes and cooled, thereby giving a sample to be electrophoresed. Western blotting analysis was performed on the sample in the same manner as the part 1-2) of Example 1, and the results are shown in FIG. 3.

As shown in FIG. 3, the composition of the present invention effectively inhibited the secretion of HRF, which is increased by C48/80. Thus, from the fact that the secretion of HRF is inhibited by the proton pump inhibitor, it can be found that the proton pump is involved in the secretion of HRF.

As a result, the inventive composition containing the proton pump inhibitor as an active ingredient is useful for the prevention and treatment of various allergy symptoms caused by HRF.

### Example 2

### Effect of Inventive Composition on Anaphylaxis Inhibition

1) Preparation of the Composition of the Present Invention

Pantoprazole (PANTOLOC, Pacific Pharaceuticals) was dissolved in a phosphate buffered saline at a concentration of 4.8 mM to prepare the composition of the present invention.

When administering the inventive composition to animals, the pantoprazole was used at the amount of 10 mg/kg body weight.

2) Effect of Inventive Composition on Anaphylaxis Inhibition

The effect of the composition of the present invention on anaphylaxis inhibition was examined as follows.

SD mice weighing 200-220 g were divided into three groups consisting of a negative control group, a positive control group and a test group, each group consisting of eight animals. In the negative and positive control groups, a phosphate buffered saline was administered by injection through the tail vein, and in the test group, the inventive composition was administered by injection through the tail vein in such a manner that the amount of use of pantoprazole was 10 mg/kg body weight.

30 minutes after this pretreatment, the negative control group was injected with a phosphate buffer saline through the tail vein, the positive control group and the test group were injected with 10 mg/kg of C48/80 through the tail vein. After the injection, the number of individuals died of anaphylaxis reaction caused by C48/80 was observed. The standard of death and survival was determined by whether or not the animals survive for one hour after C48/80 administration. The result of mortality for each group is given in Table 1 below.

TABLE 1

| | | | Mortality |
|---|---|---|---|
| | | Anaphylaxis | (number of |
| | | reaction- | dead individuals/ |
| | Pretreatment | causing | number of |
| Groups | (−30 minutes) | substance | total individuals) |
| Negative | Phosphate | Phosphate | 0/8 |
| control group | buffered saline | buffered saline | |
| Positive | | C48/80 | 7/8 |
| control group | | (10 mg/kg) | |
| Test group | Inventive | | 4/8 |
| | composition | | |
| | (10 mg/kg) | | |

As shown in Table 1, it can be found that the composition of the present invention remarkably reduces anaphylaxis reaction in the animals. This result coincides with the result of Example 1 showing that the inventive composition inhibits the secretion of HRF from cells.

As a result, the composition of the present invention is useful for the prevention and treatment of diseases caused by a hypersensitive immune response which can be induced by HRF.

3) Effect of Inventive Composition on Blood Histamine Concentration

In order to examine if the effect of the inventive composition on anaphylaxis inhibition, as indicated the part 2) of Example 2, is attributed to the reduction of blood histamine concentration, the following test was performed.

In the case of the dead mice among the mice used in the part 2 of Example 2, a small amount of blood was collected from the heart just before or just after death. In the case of the survived mice, a small amount of blood was collected after anesthesia with ether.

The collected blood was left to stand at 4° C. overnight to coagulate blood-corpuscles, and centrifuged at 3,000 rpm for 10 minutes, and the serum was isolated. A portion of the serum was taken, 10 to 300-fold diluted in deionized water, and analyzed for blood histamine concentration using a histamine analyzer, and the results are given in Table 2 below.

TABLE 2

| | | Blood histamine |
|---|---|---|
| | | concentration |
| Groups | Pretreatment | (average; ng/ml) |
| Negative control group | Phosphate buffered saline | Not measurable* |
| Positive control group | | 2646.47 |
| Test group | Inventive composition | 301.8 |
| | (10 mg/kg) | |

*not measurable: concentration was lower than a measurable range.

As shown in Table 2, in the blood of the test group pretreated with the inventive composition, the amount of histamine release was at least 80% lower than the positive control group.

As a result, the inventive composition has the effect of reducing blood histamine level and thus is useful for the prevention and treatment of anaphylaxis reaction.

4) Effect of Inventive Composition on Blood HRF Concentration

In order to examine if the effect of the inventive composition on the reduction of blood histamine concentration, as indicated in the part 2) of Example 2, is attributed to the reduction of HRF secretion, the following test was conducted.

The serum of each group obtained in the part 3) of Example 2 was mixed with the same amount of 4× sample buffer solution. At this time, the ratio of glycerol in the used sample buffer solution was 50% such that the attraction phenomenon of bands upon electrophoresis is prevented. The sample mixture was denatured in boiling water for 3 minutes, cooled on ice for a short time, and centrifuged at 12,000 rpm for 5 minutes, and only the supernatant was collected.

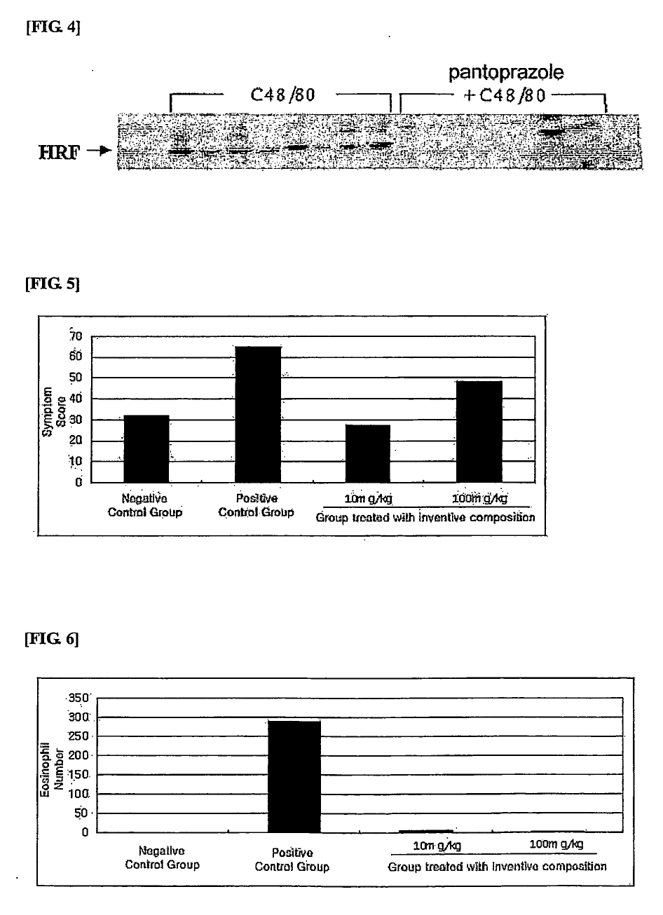

In the same manner as the part 1-2) of Example 1, Western blotting analysis was conducted for the sample prepared as described above, and the results are shown in FIG. 4.

As shown in FIG. 4, the band of HRF was obviously detected in most of the blood of the animals that were not pretreated with the inventive composition and thus died of anaphylaxis reaction.

On the other hand, the band of HRF was not substantially detected in the blood of the group that was pretreated with the inventive composition and thus survived.

As a result, it can be found that HRF plays an important role in anaphylaxis reaction caused by C48/80, and the composition of the present invention can reduce blood HRF concentration and thus blood histamine level, thereby inhibiting anaphylaxis reaction.

### Example 3

### Effect of Inventive Composition on Inhibition of Allergic Rhinitis

1) Preparation of Inventive Composition

When treating animals with the inventive composition, the composition was dissolved in a phosphate buffered saline such that pantoprazole is applied at the amounts of 10 mg/kg and 100 mg/kg for two test groups, respectively.

2) Test Method

3-week-old BALBc male mice weighing 15-25 g were divided into four groups each comprising 6 or 7 animals, and then bred in a specific pathogen-free (SPF) environment for 35 days.

In order to induce allergic rhinitis, 10 μg of ovalbumin (grade V, Sigma Chemical Co., Phillipsberg, N.Y.) and 1 mg of aluminum hydroxide (Al(OH)₃) gel were mixed with 0.3 ml phosphate buffered saline for at least 30 minutes to prepare an ovalbumin mixture.

0.1 ml of the mixture was injected into the abdominal cavity of the mice, and each at 5, 14 and 21 days later, injected once more into the abdominal cavity. At one week after the final injection into the abdominal cavity (28 days later), 30 μl phosphate buffered saline or a mixture of 100 μg ovalbumin with 30 μl phosphate buffered saline was administered into the nasal cavity using a micropipette. At 30 minutes before the administration into the nasal cavity, a phosphate buffered saline and 10 mg/kg and 100 mg/kg of pantoprazole were administered into the abdominal cavity of control groups and two test groups, respectively, and the effect of the inventive composition on the inhibition of allergic rhinitis was examined.

The administration of ovalalbumin into the nasal cavity and the pretreatment with the drugs before this administration were performed for 7 days, one time a day, and the next day after the administration for 7 days, the mice of all the groups was euthanized by inhalation with carbonic acid gas. The test procedure as described above is summarized in Table 3 below.

TABLE 3

| | | Systemic senitization | | | | 30 minutes before | | Euthanasia |
|---|---|---|---|---|---|---|---|---|
| Groups | Number | 1 day | 5 days | 14 days | 21 days | causing rhinitis | 28-24 days | 35 days |
| Negative | 6 | OVA + | OVA + Alum | OVA + Alum | OVA + | Phosphate buffered | Phosphate | |
| control group | | Alum | | | Alum | saline | buffered saline | |
| Positive | 6 | | | | | | OVA | |
| control group | | | | | | | | |
| Test group I | 7 | | | | | 10 mg/kg of | | |
| | | | | | | inventive composition | | |
| Test group II | 7 | | | | | 100 mg/kg of | | |
| | | | | | | inventive. | | |
| | | | | | | composition | | |

| | | | |
|---|---|---|---|
| Remarks | Mixture of 10 μg OVA and 1 mg Alum | Pretreatment before | Phosphate or |
| | was dissolved 300 μl phosphate | administration of | mixture of 100 μg |
| | buffered saline and administered into | OVA into nasal cavity | and 30 μl |
| | abdominal cavity | | phosphate |
| | | | buffered saline |

(OVA: ovalbumin; and Alum: Al(OH)₃)

3) Effect of Inventive Composition on Inhibition of Symptoms of Allergic Rhinitis

The effect of the inventive composition on the inhibition of symptoms of allergic rhinitis was examined as follows:

The induction of allergic rhinitis in the nasal cavity was conducted in the same manner as the part 2) of this Example. Then, in each mouse, the number of expression of characteristic symptoms of allergic rhinitis, i.e., the number of expression of symptoms, such as sneezing and the action of scratching the nose, was measured for 15 minutes after a given period, and their sum was recorded as a symptom score. The symptom score was recorded as the mean of scores measured by two observers, and scores having at least 30% difference between the two observers were excluded. The symptom score for each group is shown in Table 4 and FIG. 5.

TABLE 4

| | | | | Group treated | Group treated |
|---|---|---|---|---|---|
| | | Negative | Positive | with inventive | with inventive |
| | | control | control | composition | composition |
| | | group | group | (10 mg/kg) | (100 mg/kg) |
| Symptom | Mean | 31.38 | 65.00 | 27.29 | 48.00 |
| score | Standard deviation | 20.84 | 33.15 | 28.49 | 17.42 |
| p-value | Negative control group | — | 0.078 | 0.391 | 0.1 |
| | Positive control group | — | — | 0.046 | 0.317 |

As shown in Table 4 and FIG. 5, it was found that in the groups treated with the inventive composition, symptoms of allergic rhinitis were remarkably reduced as compared to the positive control group.

As a result, it can be found that the inventive composition can effectively reduce allergic diseases, including allergic rhinitis and hay fever.

4) Histological Examination

The effect of the inventive composition on the increase of eosinophil number that is the histological characteristic of allergic diseases was examined as follows.

The head of the mice enthanized in the part 2) of this Example 2 was separated and immobilized in 10% formalin solution for 24 hours. Then, the skin was removed from the mouse's head and immersed in decalcifier B (Accra Lab, Swedesboro, N.J.) for 24 hours. The nasal tissue was embedded in paraffin and then a coronal section with a 5 μm thickness was collected.

This section was dyed with haematoxyloin and eosin, and then, a tissue showing two teeth in the coronal section of the mouse nasal cavity was selected under an optical microscope and measured for eosinophil number under a mucosa in the anterior portion of the inferior turbinate at 400× magnifications. The measured results were statistically analyzed by the Mann-Whitney U-test, using SPSS for Windows version 10 (SPSS Inc., Chicago), and were determined to be statistically significant at p<0.05. The results are shown in Table 5 and FIG. 6.

TABLE 5

| | | Negative | Positive | Group administered with | Group administered with |
|---|---|---|---|---|---|
| | | control | control | inventive composition | inventive composition |
| | | group | group | (10 mg/kg) | (100 mg/kg) |
| Symptom | Mean | 1.33 | 290.00 | 5.57 | 4.29 |
| score | | | | | |
| | Standard deviation | 2.34 | 73.63 | 13.00 | 4.99 |
| p-value | Negative control group | — | 0.004 | 0.703 | 0.366 |
| | Positive control group | — | — | 0.003 | 0.003 |

As shown in Table 5 and FIG. 6, the groups administered with the composition of the present invention showed a very lower eosinophil number than the positive control group.

As a result, it can be found that the composition of the present invention can effectively inhibit allergic diseases, including allergic rhinitis and hay fever.

### INDUSTRIAL APPLICABILITY

As described above, the composition of the present invention has the effect of inhibiting HRF secretion induced by allergy-causing substances.

Furthermore, the composition of the present invention has the effect of reducing the amount of HRF and histamine in blood, so as to remarkably reduce the probability of death from systemic anaphylaxis reaction induced by the allergy-causing substances and to mitigate symptoms of allergic rhinitis.

Accordingly, the composition of the present invention can be advantageously used as a pharmaceutical composition for the prevention and treatment of various allergic diseases caused by HRF, i.e., asthma diseases, including asthma induced by foods or drugs (e.g., aspirin), allergic asthma, urticaria, anaphylaxis, allergic rhinitis, allergic bronchiectasis, hay fever, atopic dermatitis and malaria.

1. A composition for inhibiting the secretion of IgE-dependent histamine-releasing factor (HRF), which contains a benzimidazolic compound having proton pump inhibitor activity, as an active ingredient.

2. The composition of claim 1, wherein the benzimidazolic compound is a lipid-soluble weak base consisting of a benzimidazole ring, a pyridine ring and a methylsulfinyl group located between them, and has a structure represented by the following formula: 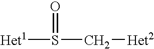
wherein Het¹ is 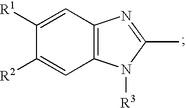
Het² is 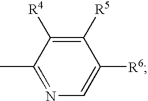
R¹ and R² which may be the same or different, each independently represents hydrogen, methoxy, or difluoromethoxy; R³ represents hydrogen or sodium; and R⁴, 5⁵ and R⁶, which may be the same or different, each independently represents hydrogen, methyl, methoxy, methoxypropoxy, or trifluoroethoxy.

3. The composition of claim 2, wherein the benzimidazolic compound is one or more selected from the group consisting of omeprazole, lansoprazole, pantoprazole, rabeprazole, and derivatives and isomers thereof.

4. The composition of claim 3, which additionally contains at least one substance selected from the group consisting of fenoctimine, oleic acid, catechin, scopadulciol, pentagalloyl glucose, bufalin, and macrolide antibiotic bafilomycin and concanamycin, which all have proton pump inhibitor activity.

5. The composition of claim 1, which is used for the prevention and treatment of various allergic diseases caused by HRF, including asthma, urticaria, anaphylaxis, allergic rhinitis, allergic bronchiectasis, hay fever, atopic dermatitis and malaria.

6. The composition of claim 5, which is used for the prevention and treatment of asthma, urticaria, allergic bronchiectasis, and atopic dermatitis.

7. The composition of claim 5, which is used for the prevention and treatment of anaphylaxis, allergic rhinitis, and hay fever.

8. The composition of claim 5, which is used for the prevention and treatment of malaria.

9. A composition for inhibiting the secretion of HRF, which contains, as a single active ingredient, at least one substance selected from the group consisting of fenoctimine, oleic acid, catechin, scopadulciol, pentagalloyl glucose, bufalin, and macrolide antibiotic bafilomycin and concanamycin, which all have proton pump inhibitor activity.

10. The composition of claim 9, which is used for the prevention and treatment of various allergic diseases caused by HRF, including asthma, urticaria, anaphylaxis, allergic rhinitis, allergic bronchiectasis, hay fever, atopic dermatitis and malaria.

11. The composition of claim 10, which is used for the prevention and treatment of asthma, urticaria, allergic bronchiectasis, and atopic dermatitis.

12. The composition of claim 10, which is used for the prevention and treatment of anaphylaxis, allergic rhinitis, and hay fever.

13. The composition of claim 10, which is used for the prevention and treatment of malaria.

14. The composition of claim 2, which is used for the prevention and treatment of various allergic diseases caused by HRF, including asthma, urticaria, anaphylaxis, allergic rhinitis, allergic bronchiectasis, hay fever, atopic dermatitis and malaria.

15. The composition of claim 3, which is used for the prevention and treatment of various allergic diseases caused by HRF, including asthma, urticaria, anaphylaxis, allergic rhinitis, allergic bronchiectasis, hay fever, atopic dermatitis and malaria.

16. The composition of claim 4, which is used for the prevention and treatment of various allergic diseases caused by HRF, including asthma, urticaria, anaphylaxis, allergic rhinitis, allergic bronchiectasis, hay fever, atopic dermatitis and malaria.
